(19) United States
(12) Patent Application Publication
(10) Pub. No.: US 2006/0087616 A1
(43) Pub. Date: Apr. 27, 2006

(54) Method and apparatus using optical coherence tomography based on spectral interference, and an ophthalmic apparatus

(75) Inventor: Masaaki Hanebuchi, Nukata-gun (JP)

Correspondence Address: OLIFF & BERRIDGE, PLC, P.O. BOX 19928, ALEXANDRIA, VA, 22320, US

(73) Assignee: NIDEK CO., LTD., Gamagori-shi (JP)

(21) Appl. No.: 11/239,152

(22) Filed: Sep. 30, 2005

(30) Foreign Application Priority Data
Sep. 30, 2004 (JP) 2004-289076
Jul. 1, 2005 (JP) 2005-194454

(51) Int. Cl. A61B 3/14

(52) U.S. Cl. 351/210

(57) ABSTRACT

A method and an apparatus using optical coherence tomography based on spectral interference where depth information of an object can be speedily obtained and an information acquisition range in a depth direction can be enlarged by removing noise, and to provide an ophthalmic apparatus. The method includes the steps of forming object light by projecting light with short coherent length onto the object, forming reference light by projecting light with short coherent length onto a reference surface, synthesizing the object and reference light to be interference light, dispersing the light into predetermined frequency components, and photo-receiving the light with a photodetector, and obtaining the depth information by subtracting respective autocorrelation signal components of the object and reference light from signal components of the photo-received interference light and performing Fourier or inverse Fourier transformation thereon, or by performing the subtraction and Fourier or inverse Fourier transformation in reverse order.

### BACKGROUND OF THE INVENTION

1. Field of the Invention

The present invention relates to a method and an apparatus for obtaining depth information of an object using optical coherence tomography (OCT) based on spectral interference, and specifically, relates to an ophthalmic apparatus for obtaining depth information of an eye.

2. Description of Related Art

Conventionally, there is known an apparatus for obtaining depth information of an object including a sectional (tomographic) image, a surface shape and a depth dimension of the object using optical coherence tomography (OCT) based on spectral interference. This kind of apparatus, which does not drive a reference mirror, can obtain the depth information of the object more speedily than a normal apparatus using optical coherence tomography (OCT) not based on spectral interference.

Such an OCT apparatus based on spectral interference obtains the depth information of the object by performing Fourier transformation on signal components of interference light (synthetic light of object light and reference light) which is dispersed into frequency components to be photo-received by a spectrometer part, allowing a sectional image of the object based on the obtained depth information to be displayed on a monitor. However, in a case where, for example, a sectional image of an anterior segment of an eye is displayed using the OCT apparatus based on spectral interference, a peak due to respective autocorrelation signal components of the object light and the reference light intensely appear in the center of a screen of the monitor, and the sectional image of the anterior segment of the eye appears as dual images flipped relative to the center line, as shown in FIG. 1A. Hence, actually, either left one or right one of the dual images shown in FIG. 1A is displayed as shown in FIG. 1B, and thereby a range capable of displaying (forming) the sectional image, i.e., an information acquisition range in a depth direction is unintentionally narrowed.

As a solution to this problem, proposed is an apparatus using an optical coherence tomography (OCT) based on spectral interference in which a position of a reference mirror is changed in phases, which allows a peak not to appear in the center of a screen, dual images not to be displayed, and a range capable of displaying (forming) a sectional image (an information acquisition range in a depth direction) to be large (see U.S. Pat. No. 6,377,349B1, DE19814057A1, and Japanese Patent Application Unexamined Publication No. Hei 11-325849). However, driving the reference mirror hinders the depth information of the object from being obtained speedily, on the contrary.

### SUMMARY OF THE INVENTION

An object of the invention is to overcome the problems described above and to provide a method and an apparatus using optical coherence tomography based on spectral interference where depth information of an object can be speedily obtained and an information acquisition range in a depth direction can be enlarged by removing noise, and to provide an ophthalmic apparatus.

To achieve the objects and in accordance with the purpose of the present invention, a method for obtaining depth information of an object using an optical coherence tomography (OCT) based on spectral interference includes the steps of forming object light which is reflection light from the object by projecting light with short coherent length thereonto, forming reference light which is reflection light from a reference surface by projecting light with short coherent length thereonto, synthesizing the object light and the reference light to be interference light, dispersing the interference light into predetermined frequency components and photo-receiving the dispersed interference light with a photodetector, and obtaining the depth information of the object by subtracting respective autocorrelation signal components of the object light and the reference light from signal components of the photo-received interference light and performing Fourier transformation or inverse Fourier transformation thereon, or performing Fourier transformation or inverse Fourier transformation on signal components of the photo-received interference light and on respective autocorrelation signal components of the object light and the reference light and subtracting the respective autocorrelation signal components of the object light and the reference light from the signal components of the photo-received interference light.

In another aspect of the present invention, an apparatus for obtaining depth information of an object using optical coherence tomography based on spectral interference includes a first projecting optical system for projecting light with short coherence length onto the object to form object light which is reflection light from the object, a second projecting optical system for projecting light with short coherence length onto a reference surface to form reference light which is reflection light from the reference surface an interference/dispersion/photo-receiving optical system for synthesizing the object light and the reference light to be interference light, dispersing the interference light into predetermined frequency components and photo-receiving the dispersed light with a photodetector, and a calculation part which obtains the depth information of the object by subtracting respective autocorrelation signal components of the object light and the reference light from signal components of the photo-received interference light and performing Fourier transformation or inverse Fourier transformation thereon, or performing Fourier transformation or inverse Fourier transformation on signal components of the photo-received interference light and on respective autocorrelation signal components of the object light and the reference light and subtracting the respective autocorrelation signal components of the object light and the reference light from the signal components of the photo-received interference light.

Additional objects and advantages of the invention are set forth in the description which follows, are obvious from the description, or maybe learned by practicing the invention. The objects and advantages of the invention may be realized and attained by the method and the apparatus in the claims.

### BRIEF DESCRIPTION OF THE DRAWINGS

The accompanying drawings, which are incorporated in and constitute a part of this specification, illustrate embodiments of the present invention and, together with the description, serve to explain the objects, advantages and principles of the invention. In the drawings,

FIGS. 1A and 1B are views showing conventional manners of image display;

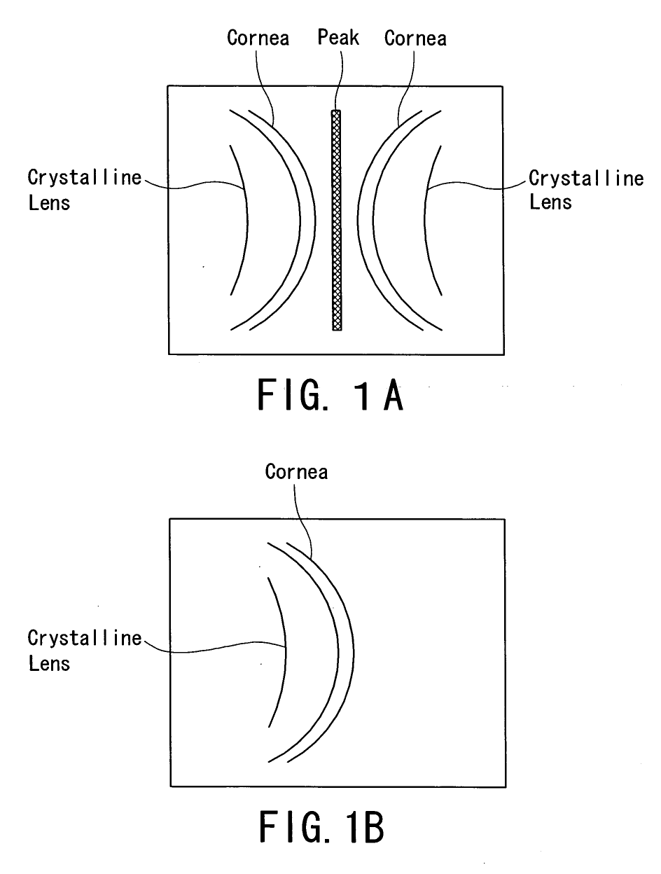

FIG. 2 is a view showing a schematic configuration of an optical system of an ophthalmic OCT apparatus based on spectral interference consistent with one preferred embodiment of the present invention;

FIG. 3 is a schematic block diagram of a control system of the ophthalmic OCT apparatus;

FIG. 4 is a view for illustrating an analytical method for obtaining a sectional image of an eye being an object;

FIG. 5 is a view showing an image corresponding to mathematical expressions employed in analysis; and

FIGS. 6A, 6B and 6C are views showing analytical procedures.

### DETAILED DESCRIPTION OF THE PREFERRED EMBODIMENTS

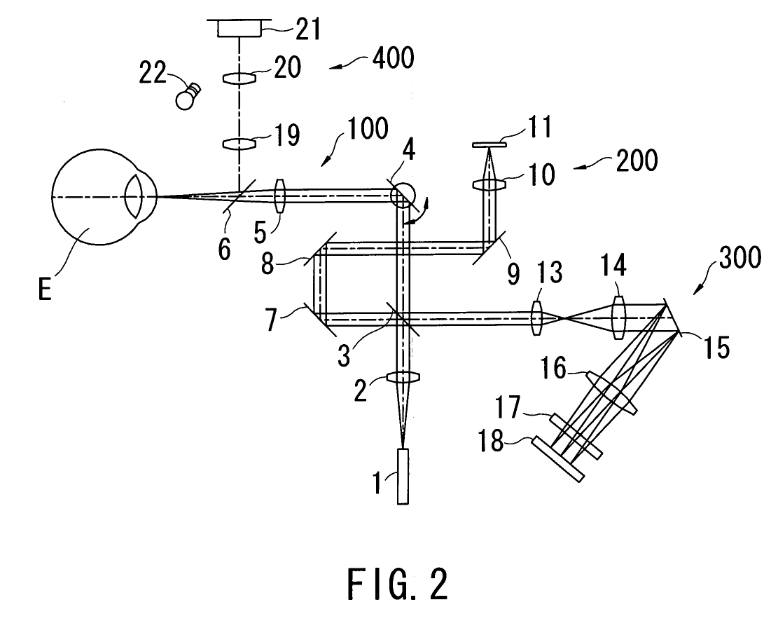

A detailed description of one preferred embodiment of a method and an apparatus using optical coherence tomography (OCT) based on spectral interference and an ophthalmic apparatus embodied by the present invention is provided below with reference to the accompanying drawings. FIG. 2 is a view showing a schematic configuration of an optical system of an ophthalmic OCT apparatus based on spectral interference consistent with the preferred embodiment of the present invention. It should be noted that the apparatus consistent with the preferred embodiment is an apparatus for picking up a sectional image of an anterior segment of an eye being an object, and its optical system includes an object-light projecting optical system, a reference-light projecting optical system, an interference/dispersion/photo-receiving optical system (an interference-signal detecting optical system), and an observation optical system. Though the apparatus consistent with the preferred embodiment includes also an alignment optical system for aligning the apparatus with the eye to have a predetermined positional relationship, a description thereof is omitted since an optical system similar to a known alignment optical system used in an objective eye refractive power measurement apparatus and the like may be employed.

<Object-Light Projecting Optical System>

An object-light projecting optical system 100 includes a light source 1, a collimator lens 2, a half mirror 3, a galvano mirror 4, an objective lens 5, and a dichroic mirror 6 which transmits near infrared light and reflects infrared light. The light source 1 such as a super luminescent diode (SLD) emits near infrared light with short coherence length. The light emitted from the light source 1 is made into parallel light by the collimator lens 2, and a part thereof passes through the half mirror 3. The light having passed through the half mirror 3 is reflected by the galvano mirror 4 and passes through the objective lens 5 and the dichroic mirror 6 to converge in the vicinity of a corneal vertex of an eye E. The galvano mirror 4 is rotated (oscillated) in a predetermined direction (in the preferred embodiment, a direction for scanning the light in an up/down direction with respect to the eye E). In addition, the galvano mirror 4 of which a reflection surface is positioned at a posterior focal point of the objective lens 5 is arranged in such a manner that an optical path length does not change.

<Reference-Light Projecting Optical System>

A reference-light projecting optical system 200 includes the light source 1, the collimator lens 2, the half mirror 3 which are shared with the object-light projecting optical system 100, total reflection mirrors 7 to 9, a condenser lens 10, and a reference mirror 11. The light from the light source 1 reflected by the half mirror 3 is reflected by the mirrors 7 to 9 and passes through the condenser lens 10 to converge at a reflection surface of the reference mirror 11.

<Interference/Dispersion/Photo-Receiving Optical System>

An interference/dispersion/photo-receiving optical system 300 includes an optical system for photo-receiving light reflected from the eye E (hereinafter also referred to as object light) and an optical system for photo-receiving light reflected by the reference mirror 11 (hereinafter also referred to as reference light).

The object-light photo-receiving optical system includes the dichroic mirror 6, the objective lens 5, the galvano mirror 4, the half mirror 3 which are shared with the object-light projecting optical system 100, a condenser lens 13, an expander lens 14, a grating mirror (diffraction grid) 15, a condenser lens 16, a cylindrical lens 17, and a photodetector 18 having sensitivity to a near infrared range. The grating mirror 15 is arranged in such a manner that its reflection surface is positioned at an anterior focal point of the condenser lens 16. In addition, the photodetector 18 is arranged in such a manner that its photo-receiving surface is positioned at a posterior focal point of the condenser lens 16.

Reflection light brought by the light which is made to converge in the vicinity of the corneal vertex of the eye E by the object-light projecting optical system 100 (i.e., the object light) passes through the dichroic mirror 6 and the objective lens 5 to be reflected by the galvano mirror 4, and a part thereof is reflected by the half mirror 3. The light reflected by the half mirror 3 passes through the condenser lens 13 to once converge, passes through the expander lens 14 to have its light bundle diameter enlarged, and enters the grating mirror 15 to be dispersed into frequency components. The light dispersed by the grating mirror 15 passes through the condenser lens 16 and the cylindrical lens 17 to converge at the photo-receiving surface of the photodetector 18. Incidentally, the light bundle diameter after the passage through the expander lens 14, grid intervals of the grating mirror 15, the condenser lens 16, and the photodetector 18 are optimized in consideration of an information acquisition range in a depth direction of the eye E (a direction of an optical axis) and a resolution thereof.

The reference-light photo-receiving optical system includes the reference mirror 11, the condenser lens 10, the mirrors 9 to 7 and the half mirror 3 which are shared with the reference-light projecting optical system 200, and the condenser lens 13, the expander lens 14, the grating mirror 15, the condenser lens 16, the cylindrical lens 17 and the photodetector 18 which are shared with the object-light photo-receiving optical system.

Reflection light brought by the light which is made to converge at the reflection surface of the reference mirror 11 by the reference-light projecting optical system 200 (i.e., the reference light) passes through the condenser lens 10 to be reflected by the mirrors 9 to 7, and a part thereof passes through the half mirror 3 to be synthesized with the object light. The reference light synthesized with the object light passes through the condenser lens 13 and the expander lens 14 to be dispersed into frequency components by the grating mirror 15, and passes through the condenser lens 16 and the cylindrical lens 17 to converge at the photo-receiving surface of the photodetector 18. In this manner, the grating mirror 15, the condenser lens 16, the cylindrical lens 17, and the photodetector 18 form a spectrometer part. Incidentally, the photodetector 18 is arranged in such a manner that its photo-receiving surface has a positional relationship conjugate with a cornea of the eye E. In addition, the cylindrical lens 17 acts to enlarge the light bundle diameter in a width direction of the photodetector 18, allowing the light to be photo-received on the photo-receiving surface of the photodetector 18 regardless of its placement error.

<Observation Optical System>

An observation optical system 400 includes the dichroic mirror 6, an objective lens 19, an image-forming lens 20, and an image-pickup element 21 having sensitivity to an infrared range. The image-pickup element 21 is arranged in such a manner that its image-pickup surface has a positional relationship conjugate with a pupil of the eye E. A light source 22 such as a light emitting diode (LED) emits infrared light and illuminates an anterior segment of the eye E. A front image of the anterior segment illuminated by the light source 22 is picked up by the image-pickup element 21 and displayed on a monitor 31.

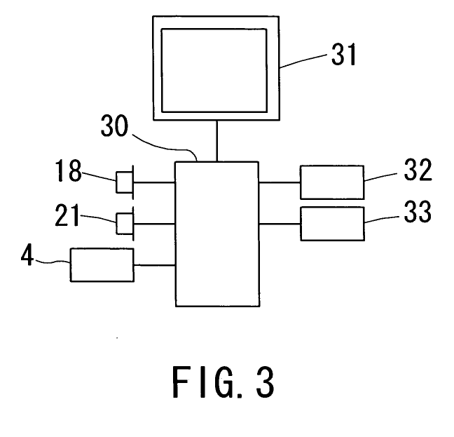

FIG. 3 is a schematic block diagram of a control system of the ophthalmic OCT apparatus. A control part 30 performs control of the entire apparatus, and the like. The control part 30 is connected with the galvano mirror 4, the photodetector 18, the image-pickup element 21, the monitor 31, a calculation/processing part 32, a storage part 33, and the like. The calculation/processing part 32 forms a sectional image of the eye E based on output signals from the photodetector 18. The storing part 33 stores the formed sectional image of the eye E.

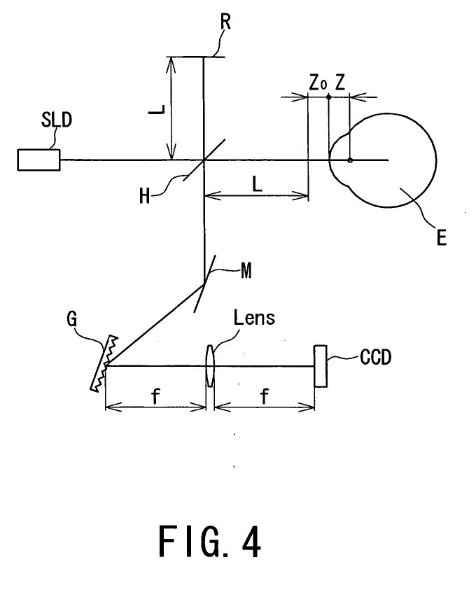

Next, with reference to FIG. 4, a description of the preferred embodiment will be given to a method (analytical method) for obtaining the sectional image of the object (the eye E in the present embodiment) based on the output signals of the light dispersed into the frequency components, which are sent from the photodetector 18. Besides, in FIG. 4, SLD denotes a light source, H denotes a half mirror (beam splitter), R denotes a reference mirror, M denotes a total reflection mirror, G denotes a grating mirror, and CCD denotes a photodetector.

An electric field of light emitted from the SLD at the H at a time t is defined as the following expression 1.
E(t)=∫_{-∞}^{∞}α_{ω}(t)e^{-i(ωt-φ(ω))}dω. Expression 1

It is expressed as integration with respect to an angular frequency ω in order to indicate the existence of wavelength distribution in the SLD. Letting 2L denote an optical path length of the light while it is reflected by the H and the R to return to the H, an electric field of reference light at the H can be expressed by the following expression 3, assuming that τᵣ is given by the following expression 2.

Besides, c denotes the speed of light.
τᵣ≡2L/c Expression 2
E_{ref}(t)=E(t−τᵣ)=∫_{-∞}²⁸ α_{ω}(t−τᵣ)e^{-i(ω(t-τ}^{ᵣ}^{)-φ(ω))}dω Expression 3
In addition, letting a corneal vertex locate further than an optical path length from the H to the R by a Z₀ portion, and letting R(Z) denote an energy reflectance at a position inside the eye E, which is located further than the corneal vertex by a Z portion, object light can be expressed by the following expression 4.

$${E}_{obj} ( t ) = {\int }_{0}^{∞} \sqrt{R ( z )} E ( t - 2 \frac{z + {z}_{0}}{C} - {τ}_{r} ) ⅆ z = \frac{1}{2} {\int }_{0}^{∞} r ( τ ) E ( t - τ - {τ}_{0} - {τ}_{r} ) ⅆ τ \qquad Expression 4$$

Here, assuming that τ₀ is given by the following expression 5,
τ₀2z₀/c′τ≡2z/c′ Expression 5
assuming that r(τ) is given by the following expression 6 in consideration of the depth direction of the phase object (eye E) is the same as a time axis of the light,
r(τ)≡2\sqrt{R(z)} Expression 6
and letting r(τ) denote an even-numbered real variable function, the expression 4 can be expanded as the following expression 7.

$$\begin{matrix}{E}_{obj} ( t ) = {\int }_{- ∞}^{∞} r ( τ ) E ( t - τ - {τ}_{0} - {τ}_{r} ) ⅆ τ \\ = {\int }_{- ∞}^{∞} r ( τ ) ⅆ τ {\int }_{- ∞}^{∞} {a}_{ω} ( t - τ - {τ}_{0} - {τ}_{r} ) \\ {ⅇ}^{- ⅈ ( ω ( t - τ - {τ}_{0} - {τ}_{r} ) - ϕ ( ω ) )} ⅆ ω \\ = {\int }_{- ∞}^{∞} r ( τ ) {E}_{ref} ( t - {τ}_{0} - τ ) ⅆ τ \\ = r ⊗ {E}_{ref} ( t - {τ}_{0} )\end{matrix} \qquad Expression 7$$

Besides, the last expression is represented using a convolution integral. For the benefit of later use, Fourier transformation is performed on the expression 7 to obtain the following expression 8, letting a sign denote Fourier transformation.

$$\begin{matrix}{\tilde{E}}_{obj} ( ω ) = {\int }_{- ∞}^{∞} {\int }_{- ∞}^{∞} r ( τ ) {E}_{ref} ( t - τ - {τ}_{0} ) ⅆ τ {ⅇ}^{- ⅈ ω t} ⅆ t \\ = [ {\int }_{- ∞}^{∞} r ( τ ) {ⅇ}^{- ⅈ ωτ} ⅆ τ ] \\ [ {\int }_{- ∞}^{∞} {E}_{ref} ( t - τ - {τ}_{0} ) {ⅇ}^{- ⅈω ( t - τ )} ⅆ t ] \\ = [ {\int }_{- ∞}^{∞} r ( τ ) {ⅇ}^{- ⅈωτ} ⅆ τ ] \\ [ {\int }_{- ∞}^{∞} {E}_{ref} ( T ) {ⅇ}^{- ⅈω ( T + {τ}_{0} )} ⅆ T ] \\ = \tilde{r} ( ω ) {\tilde{E}}_{ref} ( ω ) {ⅇ}^{- ⅈω {τ}_{0}}\end{matrix} \qquad Expression 8$$

While the reference light and the object light are made coaxial to interfere with each other at the H, the light is dispersed by a spectrometer consisting of the G, a lens and the CCD, so that interference spectrum patterns of respective wavelength components are produced on the CCD. Accordingly, on interference spectrums, Fourier transformation is performed to obtain the following expression 9. Besides, * denotes a complex conjugate.

$$\begin{matrix}\tilde{I} ( ω ) = {\vert {\tilde{E}}_{ref} ( ω ) + {\tilde{E}}_{obj} ( ω ) \vert}^{2} \\ = {\vert {\tilde{E}}_{ref} ( ω ) \vert}^{2} + {\vert {\tilde{E}}_{obj} ( ω ) \vert}^{2} + \\ {\tilde{E}}_{ref} ( ω ) \cdot {{\tilde{E}}_{obj} ( ω )}^{*} + {{\tilde{E}}_{ref} ( ω )}^{*} \cdot {\tilde{E}}_{obj} ( ω ) \\ = {\vert {\tilde{E}}_{ref} ( ω ) \vert}^{2} + {\vert {\tilde{E}}_{obj} ( ω ) \vert}^{2} + \\ {\vert {E}_{ref} ( ω ) \vert}^{2} [ {\tilde{r} ( ω )}^{*} {ⅇ}^{ⅈ ω {τ}_{0}} + \tilde{r} ( ω ) {ⅇ}^{- ⅈ ω {τ}_{0}} ]\end{matrix} \qquad Expression 9$$

While a diffraction angle made by the G is proportional to a minute deviation amount of the wavelength, the expression 9 is represented as a function of the angular frequency ω corresponding to Fourier transformation relating to time. This is because, according to the following expression 11, which is obtained by differentiating the following expression 10, a proportionality relation between the diffraction angle and a minute angular frequency deviation is established via the wavelength. Besides, f denotes a focal length, and λ denotes a frequency.

$$\begin{matrix}ω = 2 π f = \frac{2 π c}{λ} & Expression 10 \\ δ ω = \frac{2 π c}{{λ}^{2}} δ λ & Expression 11\end{matrix}$$

Depth information r(t) of the phase object is obtained by performing inverse Fourier transformation on the interference spectrums produced on the CCD with respect to ω, and intensity I(t) is expressed by the following expression 12. Besides, A denotes autocorrelation. In addition, since r(t) is assumed to denote the even-numbered real variable function, the following expression 13 holds.

$$\begin{matrix}\begin{matrix}I ( t ) = A [ {E}_{ref} ( t ) ] + A [ {E}_{obj} ( t ) ] + A [ {E}_{ref} ( t ) ] ⊗ \\ [ {\int }_{- ∞}^{∞} {\tilde{r} ( ω )}^{*} {ⅇ}^{ⅈ ω {τ}_{0}} {ⅇ}^{ⅈ ω t} ⅆ ω + \\ {\int }_{- ∞}^{∞} \tilde{r} ( ω ) {ⅇ}^{ⅈ ω {τ}_{0}} {ⅇ}^{- ⅈω t} ⅆ ω ] \\ = A [ {E}_{ref} ( t ) ] + A [ {E}_{obj} ( t ) ] + A [ {E}_{ref} ( t ) ] ⊗ \\ [ {( {\int }_{- ∞}^{∞} \tilde{r} ( ω ) {ⅇ}^{ⅈω ( - t - {τ}_{0} )} ⅆ ω )}^{*} + \\ {\int }_{- ∞}^{∞} \tilde{r} ( ω ) {ⅇ}^{ⅈ ω ( t - {τ}_{0} )} ⅆ ω ] \\ = A [ {E}_{ref} ( t ) ] + A [ {E}_{obj} ( t ) ] + A [ {E}_{ref} ( t ) ] ⊗ \\ [ {r ( - t - {τ}_{0} )}^{*} + r ( t - {τ}_{0} ) ] \\ = A [ {E}_{ref} ( t ) ] + A [ {E}_{obj} ( t ) ] + A [ {E}_{ref} ( t ) ] ⊗ \\ r ( - t - {τ}_{0} ) + A [ {E}_{ref} ( t ) ] ⊗ r ( t - {τ}_{0} )\end{matrix} & Expression 12 \\ r ( t ) = {r ( t )}^{*} = r ( - t ) & Expression 13\end{matrix}$$

In the expression 12, the first and the second terms respectively represent autocorrelation functions of the reference light and the object light, and the third and the forth terms represent the depth information of the phase object to obtain by expressing the autocorrelation function of the reference light as a point response function.

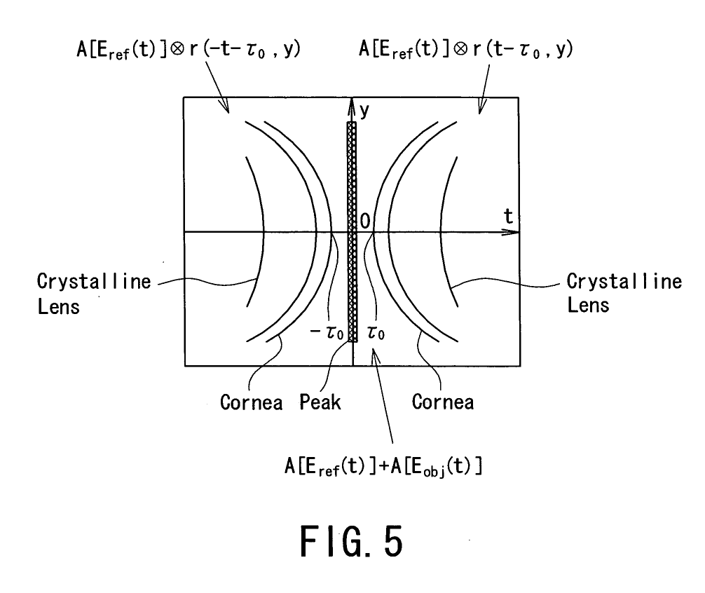

Incidentally, in the case of using the optical system shown in FIG. 2 where the eye E is regarded as the phase object, and carrying out an analysis based on the output signals from the photodetector 18 of the spectrometer part using the expression 12, an image shown in FIG. 5 is obtained. In the expression 12, the first and the second terms are the peak in the FIG. 5, and the forth term represents r(t) which is moved to the + side by τ₀ portion, and the third term represents the forth term which is flipped at an axis where t is zero. Besides, in the above description, it is assumed that inverse Fourier transformation is performed on the expression 9 to obtain the expression 12; however it is not limited hereto, and performing either Fourier transformation or inverse Fourier transformation on the expression 9 similarly allows the depth information of the phase object to be obtained since performing Fourier transformation on the expression 9 only makes a difference that variables of the respective autocorrelation functions of the reference light and the object light are inverted.

In addition, since r(t) is assumed to denote the even-numbered real variable function in the expression 13, the following expression 14 holds.

$$\begin{matrix}{\tilde{r} ( ω )}^{*} = {[ {\int }_{- ∞}^{∞} r ( t ) {ⅇ}^{- ⅈ ω t} ⅆ t ]}^{*} \\ = {\int }_{- ∞}^{∞} {r ( t )}^{*} {ⅇ}^{ⅈ ω t} ⅆ t \\ = {\int }_{- ∞}^{∞} r ( - t ) {ⅇ}^{ⅈ ω t} ⅆ t \\ = {\int }_{- ∞}^{∞} r ( t ) {ⅇ}^{- ⅈ ω t} ⅆ t \\ = \tilde{r} ( ω )\end{matrix} \qquad Expression 14$$

Accordingly, the expression 9 is rearranged to be the following expression 15.

$$\begin{matrix}\tilde{I} ( ω ) = {\vert {\tilde{E}}_{ref} ( ω ) \vert}^{2} + {\vert {\tilde{E}}_{obj} ( ω ) \vert}^{2} + \\ {\vert {\tilde{E}}_{ref} ( ω ) \vert}^{2} \tilde{r} ( ω ) [ {ⅇ}^{ⅈ ω {τ}_{0}} {ⅇ}^{- {ⅈωτ}_{0}} ] \\ = {\vert {\tilde{E}}_{ref} ( ω ) \vert}^{2} + {\vert {\tilde{E}}_{obj} ( ω ) \vert}^{2} + \\ 2 {\vert {\tilde{E}}_{ref} ( ω ) \vert}^{2} \tilde{r} ( ω ) cos ( ω {τ}_{0} )\end{matrix} \qquad Expression 15$$

In the expression 15, if cos(ωτ₀) can be made zero (in other words, a contribution of a signal from the object becomes zero), only respective autocorrelation signal components of the reference light and the object light (i.e., peak signals becoming noise) remain. Then, the remainders are removed from the expression 9 or the expression 12, allowing an image from which the peak is removed to be obtained (in the present embodiment, obtained is a sectional image of an anterior segment of an eye).

Incidentally, Υ₀ represents an unequal optical path difference (time) between the reference light and the object light. Υ₀ can be obtained by finding, through image processing, a distance from the center of the image to a vertex position in a depth direction shape of the object (in the present embodiment, the vertex position is the corneal vertex which is the foreground of the eye E being the object) which is obtained by the expression 12, and by converting the obtained distance into time. Besides, in the present embodiment, Υ₀ is obtained by finding the distance from the image center to the corneal vertex through image processing; however it is not limited hereto. For example, Υ₀ can be also obtained by additionally providing a mechanism which detects a working distance and using a detection result of the working distance. In addition, in the case of obtaining a two-dimensional sectional image of an eye, each Υ₀ can be obtained with respect to the two-dimensional sectional image based on corneal curvature which is obtained by an existing corneal shape measurement apparatus (i.e., curvature corresponding to the sectional image to obtain) and the corneal vertex which is previously obtained.

Once Υ₀ is obtained, returning to the expression 15, only respective power spectrums of the reference light and the object light can be obtained (presumed) from interference intensity on the CCD having an angular frequency ω by which cos(ωΥ₀) is made zero. Then, by subtracting the obtained power spectrums from the expression 9 and performing Fourier transformation (or inverse Fourier transformation) thereon, the autocorrelation signal components becoming noise are removed, allowing only the depth information of the phase object to be obtained. Besides, the respective power spectrums of the reference light and the object light may be subtracted after performing Fourier transformation.

The interference intensity distribution on the CCD at the stage where the respective power spectrums of the reference light and the object light have been subtracted therefrom can be expressed by the following expression 16.
Ĩ(ω)=2|\tilde{E}_{ref}(ω)|²\tilde{r}(ω)cos(ωτ₀) Expression 16
By multiplying the expression 16 by −tan(ωτ₀) using the already known τ₀ which is described above, the following expression 17 is given.
Ĩ(ω)′=Ĩ(ω)×−tan(ωΥ₀)=−2|\tilde{E}_{ref}(ω)|²\tilde{r}(ω)sin(ωτ₀) Expression 17
Here, the following expression 18 is given, assuming that it has the expression 16 as a real part and the expression 17 as an imaginary part.
Ĩ(ω)+iĨ(ω)′=2|\tilde{E}_{ref}(ω)|²\tilde{r}(ω)e^{-iωτ}^{₀} Expression 18
Then, on the expression 18, inverse Fourier transformation is performed to obtain the following expression 19.
∫_{-∞}^{∞}{Ĩ(ω)−iĨ(ω)′}e^{fωt}dw=2A[E_{ref}(t)]⊕∫_{-∞}^{∞}\tilde{r}(ω)^{-iωτ}^{₀}e^{iωt}=2A[E_{ref}(t)]⊕∫_{-∞}^{∞}\tilde{r}(ω) e^{iω(t-τ}^{₀}⁾dω2A[E_{ref}(t)]⊕r(t−τ₀) Expression 19
In the obtained expression 19, in which the terms representing the respective autocorrelation functions of the reference light and the object light have already disappeared, the term representing the depth information of the phase object becoming a virtual image disappears; therefore employing the expression 19 allows only the depth information of the phase object becoming a real image to be obtained.

By employing this method (analytical method), the information acquisition range in the depth direction can be enlarged since the image on the screen is not discarded by either right half or left half. Besides, a sectional image of the phase object which is obtained as dual images symmetrical with respect to the center line may be converted into a proper image (single image) by image processing. The image processing is performed such that either of the dual images is flipped to be super imposed on the other one regarding an image obtained thereby as the proper image, or either of the dual images is deleted, whereby a desired image is obtained.

Hereinafter, an operation of the apparatus with the aforementioned configuration will be described.

While observing the front image of the anterior segment of the eye E illuminated by the light source 22 which is displayed on the monitor 31, an examiner moves the apparatus in up/down, left/right and back/forth directions using operating means such as a joystick not illustrated and aligns the apparatus to have a predetermined positional relationship with the eye E. Besides, in the preferred embodiment, the alignment is performed so that the image-pickup surface of the image-pickup element 21 and the pupil of the eye E have a conjugate positional relationship. Incidentally, in FIG. 2, the corneal vertex is set as the reference position for obtaining the depth information. Since the information acquisition range in the depth direction is a predetermined range in the back/forth direction from the reference position, the above-described optical path difference τ₀ between the reference light and the object light should be made to be τ₀<0 in a case where the sectional image is desired to be obtained in a range as wide as possible.

When the apparatus is brought to have the predetermined positional relationship with the eye E, the examiner operates a switch not illustrated to display the sectional image of the anterior segment of the eye E on the monitor 31.

In other words, the switch not illustrated being pressed, the control part 30 controls to emit the light from the light source 1 and rotate the galvano mirror 4 to scan the light with respect to the eye E. The reflection light brought by the light which is made to converge in the vicinity of the corneal vertex of the eye E by the object-light projecting optical system 100 (i.e., the object light) and the reflection light brought by the light which is made to converge at the reflection surface of the reference mirror 11 by the reference-light projecting optical system 200 (i.e., the reference light) are synthesized by the half mirror 3 to be interference light. Then, the interference light passes through the condenser lens 13 and the expander lens 14 and enters the grating mirror 15 to be dispersed into the frequency components. The dispersed light passes through the condenser lens 16 and the cylindrical lens 17 to converge at the photo-receiving surface of the photodetector 18.

The photodetector 18 photo-receives the light dispersed into the frequency components and outputs interference strength for each frequency component as a signal. The calculation/processing part 32 monitors the output signal (interference strength) from the photodetector 18. Incidentally, the light photo-received on the photodetector 18 includes not only the reflection light from an anterior surface of the cornea (i.e., the object light) but also reflection light from a posterior surface of the cornea, anterior/posterior surfaces of a crystalline lens, and the like (i.e., the object light). Accordingly, interference light of this reflection light (i.e., the object light) and the reference light is photo-received on the photodetector 18 as a function of frequency.

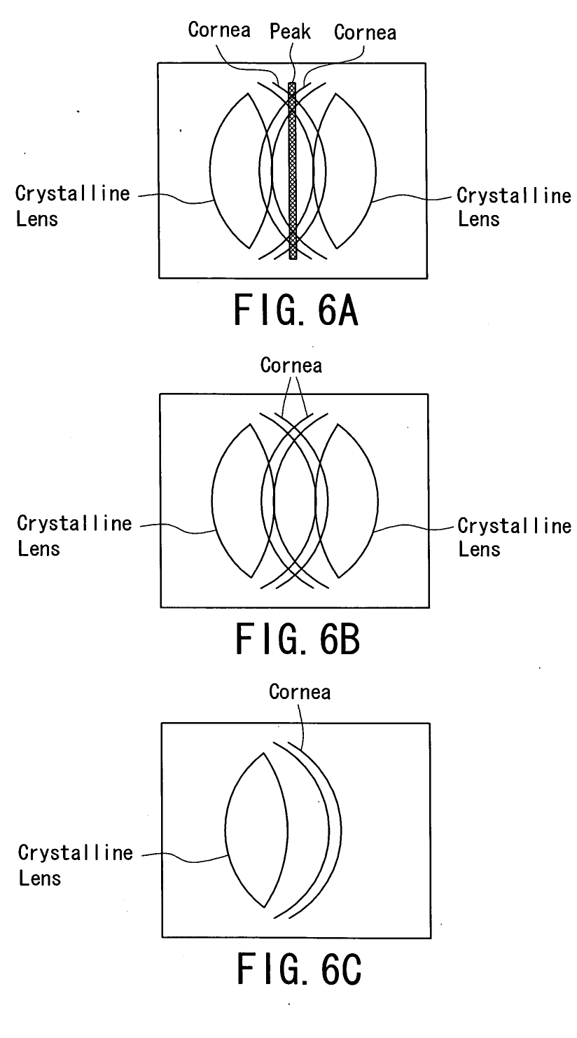

The calculation/processing part 32 performs the above-described Fourier transformation to analyze the output signal from the photodetector 18 at the time when the interference strength is maximized. Since the interference light includes the reflection light from respective phase objects of the eye E (e.g., the anterior/posterior surfaces of the cornea, the anterior/posterior surfaces of the crystalline lens, and the like) (i.e., the object light), Fourier transformation on the output signal from the photodetector 18 enables obtaining depth information on the respective phase objects such as the cornea and the crystalline lens of the eye E. The calculation/processing part 32 ordinarily removes the peak signals becoming noise (autocorrelation signal components) from data which forms the peak signals and the dual images shown in FIG. 6A using the above-described analytical method, so as to obtain an image shown in FIG. 6B. Further, using the above-described analytical method, the sectional image becoming a virtual image is removed to eventually obtain a sectional image of the anterior segment of the eye E shown in FIG. 6C, which is displayed on the monitor 32. Besides, by performing image processing by which either of the dual images is deleted, or flipped to be superimposed on the other one, the sectional image of the anterior segment of the eye E shown in FIG. 6C may be eventually obtained.

In the above preferred embodiment, described is a method by which the peak signals becoming noise are removed to allow the information acquisition range of the depth direction to be enlarged. Hereinafter, as the second preferred embodiment, a method for obtaining an image with a sharp edge will be described. Incidentally, a description of respective configurations of an optical system, a control system and an operation of an apparatus, being the same as those in the above-described preferred embodiment, is omitted, and a detailed description will be given to an analytical method for obtaining an image. In addition, signs used in the following expressions have the same meanings as above-mentioned ones as far as no particular reference is made thereto.

The interference intensity distribution on the photodetector 18 shown in FIG. 2 can be rearranged to be the following expression 20 based on the above-described expressions 8 and 9.
Ĩ(ω)=|\tilde{E}_{ref}(ω)|²[1+|\tilde{r}(ω)|²+\tilde{r}(ω)*e^{iωτ}^{₀}+\tilde{r}(ω)e^{-iωτ}^{₀}] Expression 20
In addition, the depth information r(t) of the phase object which is obtained by performing Fourier transformation or inverse Fourier transformation on the expression 20 can be expressed as the following expression 12 which is already described above.
I(t)=A[E_{ref}(t)]+A[E_{obj}(t)]+A[E_{ref}(t)]⊕r(−t−τ₀)+A[E_{ref}(t)]⊕r(t−τ₀) Expression 12

According to the expression 12, the depth information of the phase object is expressed such that the autocorrelation of the reference light is integrated by a convolution operation, causing inadequacy in resolution accordingly. In the present embodiment, in order to avoid such inadequacy, a power spectrum of the reference light expressed as the following expression 21 is found in advance, and then by the expression 21, the expression 20 is divided to obtain the following expression 22.
|\tilde{E}_{ref}(ω)|² Expression 21
Ĩ(ω)″=1+\tilde{r}(ω)|²+\tilde{r}(ω)*e^{iωτ}^{₀}+\tilde{r}(ω)e^{-iωτ}^{₀}
On the obtained expression 22, inverse Fourier transformation or Fourier transformation is performed to obtain the following expression 23.

$$\begin{matrix}{\int }_{- ∞}^{∞} {\tilde{I} ( ω )}^{″} ⅆ ω = {\int }_{- ∞}^{∞} \{ 1 + {\vert \tilde{r} ( ω ) \vert}^{2} + {\tilde{r} ( ω )}^{*} {ⅇ}^{ⅈ ω {τ}_{0}} + \\ \tilde{r} ( ω ) {ⅇ}^{- ⅈ ω {τ}_{0}} \} {ⅇ}^{ⅈω t} ⅆ ω \\ = δ ( t ) + A [ r ( t ) ] + r ( - t - {τ}_{0} ) + r ( t - {τ}_{0} )\end{matrix} \qquad Expression 23$$

The obtained expression 23 is such that the information of the autocorrelation is removed in advance; therefore the actual depth information of the phase object is not affected by the autocorrelation of the light source. Consequently, the image obtained using the expression 23 becomes sharp. Incidentally, a method to find the power spectrum of the reference light is described as follows.

According to Wiener-Khinchine's theorem, it is known that a power spectrum representing wave energy of an angular frequency ω (i.e., a square of an absolute value of the original function on which Fourier transformation is performed, which is represented by the expression 21) is obtained by performing Fourier transformation on an autocorrelation function, and the autocorrelation function is conversely obtained by performing inverse Fourier transformation on the power spectrum.

In addition, assuming that Γ env(t(ω)) denotes a function of an envelope of a coherent function (i.e., coherent time, half breadth of which corresponds to coherent length) the autocorrelation of the reference light is expressed as the following expression 24.
A[E_{ref}(t)]=Γₑₙᵥ(t(ω))e^{-iωt} Expression 24
Accordingly, the following expression 25 holds.
|\tilde{E}_{ref}(ω)|²=FT[A[E_{ref}(t)]]=FT[Γₑₙᵥ(t(ω))e^{-ωt}] Expression 25

Incidentally, the coherent function can be obtained in advance from a result which is quantitatively obtained with respect to the light emitted from the in-use light source (i.e., light with short coherent length) using an interferometer or the like. By substituting the obtained coherent function into the expression 25 and performing Fourier transformation thereon, the power spectrum of the reference light can be obtained. In addition, it is possible to previously store several types of power spectrums of the reference light in the storing part of the apparatus according to use conditions of the light source such as a temperature and an electric current, and to select the power spectrums stored in the storing part using setting means not illustrated or automatically in accordance with actual use conditions of the light source.

In addition, by taking both the analytical methods employed in the first and the second preferred embodiments into consideration, the information acquisition range in the depth direction can be enlarged while controlling noise, to say nothing of the ability to obtain a sharp image.

Incidentally, though the light to be the object light is made to converge in the vicinity of the corneal vertex of the eye E in the preferred embodiments, the present invention is not limited thereto. It is essential only that the reflection light from the phase objects of the eye (the cornea, the crystalline lens, and the like) be dispersed into the frequency components and photo-received on the photodetector. For example, the light to be the object light may be made to converge in the vicinity of the pupil of the eye.

In addition, though the grating mirror (diffraction grid) is used as dispersing means for dispersing the synthetic light of the object light and the reference light into the frequency components in the preferred embodiments, the present invention is not limited thereto. Other dispersing means such as a prism and an acoustic optical element may be employed.

In addition, though the ophthalmic OCT apparatus consistent with the preferred embodiments is an apparatus for picking up a sectional image of an anterior segment of an eye, the present invention is not limited thereto and may be applied to, for example, an apparatus for measuring a surface shape, a depth dimension such as an axial length, and the like of the eye. It goes without saying that the present invention may be applied to an apparatus for picking up a sectional image of a phase object other than the phase objects of the eye in other fields than an ophthalmologic field.

Incidentally, in the above-described analytical methods, the respective power spectrums of the object light and reference light are obtained from interference intensity on the CCD having the angular frequency ω by which cos(ωτ₀) is made zero, which is obtained based on the unequal optical path difference (time) τ₀ between the object light and reference light, or the power spectrum of the reference light obtained using the coherent function is used for removing the autocorrelation signal components; however the present invention is not limited thereto. By designing an optical system in which object light and reference light can be photo-received on a photodetector while dispersed separately, obtaining respective power spectrums of the object light and the reference light, and subtracting the first and the second terms and dividing the third term in the above-described expression 9 using the obtained respective power spectrums of the object light and reference light, the autocorrelation signal components can be removed from the expression 9. In addition, the obtained power spectrum of the reference light may be applied to the above-described expression 22. Additionally, in a case where the autocorrelation signal component of the object light is so small as to be insensitive to that of the reference light, it is negligible.

The foregoing description of the preferred embodiments of the invention has been presented for purposes of illustration and description. It is not intended to be exhaustive or to limit the invention to the precise form disclosed, and modifications and variations are possible in the light of the above teachings or may be acquired from practice of the invention. The embodiments chosen and described in order to explain the principles of the invention and its practical application to enable one skilled in the art to utilize the invention in various embodiments and with various modifications as are suited to the particular use contemplated. It is intended that the scope of the invention be defined by the claims appended hereto, and their equivalents.

What is claimed is:

1. A method for obtaining depth information of an object using an optical coherence tomography based on spectral interference, comprising the steps of:
forming object light which is reflection light from the object by projecting light with short coherent length thereonto;
forming reference light which is reflection light from a reference surface by projecting light with short coherent length thereonto;
synthesizing the object light and the reference light to be interference light, dispersing the interference light into predetermined frequency components, and photo-receiving the dispersed interference light with a photodetector; and
obtaining the depth information of the object by one of:
subtracting respective autocorrelation signal components of the object light and the reference light from signal components of the photo-received interference light, and performing Fourier transformation or inverse Fourier transformation thereon; and
performing Fourier transformation or inverse Fourier transformation on signal components of the photo-received interference light and on respective autocorrelation signal components of the object light and the reference light, and subtracting the respective autocorrelation signal components of the object light and the reference light from the signal components of the photo-received interference light.

2. The method according to claim 1, wherein the respective autocorrelation signal components of the object light and the reference light are obtained based on interference intensity on the photodetector having a plurality of angular frequencies by which contributions of signals from the object are made zero.

3. The method according to claim 2, wherein the angular frequencies by which the contributions of the signals from the object are made zero are obtained based on an unequal optical path difference between the object light and the reference light.

4. The method according to claim 3, wherein
with respect to the signal components of the photo-received interference light from which the respective autocorrelation signal components of the object light and the reference light have been subtracted, signal components with a phase changed by 90 degrees based on the unequal optical path difference between the object light and the reference light are obtained, and
Fourier transformation or inverse Fourier transformation is performed on a combination of the signal components of the photo-received interference light from which the respective autocorrelation signal components of the object light and the reference light have been subtracted, and the obtained signal components where the phase is changed.

5. The method according to claim 1, wherein
the object light and the reference light are dispersed separately to be photo-received on the photodetector, and
the respective signal components of the photo-received object light and reference light are taken as the respective autocorrelation signal components.

6. The method according to claim 1, wherein
the object is an eye, and
at least one of a sectional image, a surface shape and a depth dimension of the eye is obtained as the depth information of the object.

7. An apparatus for obtaining depth information of an object using optical coherence tomography based on spectral interference, the apparatus comprising:
a first projecting optical system for projecting light with short coherence length onto the object to form object light which is reflection light from the object;
a second projecting optical system for projecting light with short coherence length onto a reference surface to form reference light which is reflection light from the reference surface;
an interference/dispersion/photo-receiving optical system for synthesizing the object light and the reference light to be interference light, dispersing the interference light into predetermined frequency components, and photo-receiving the dispersed interference light with a photodetector; and
a calculation part which obtains the depth information of the object by one of:
subtracting respective autocorrelation signal components of the object light and the reference light from signal components of the photo-received interference light, and performing Fourier transformation or inverse Fourier transformation thereon; and
performing Fourier transformation or inverse Fourier transformation on signal components of the photo-received interference light and on respective autocorrelation signal components of the object light and the reference light, and subtracting the respective autocorrelation signal components of the object light and the reference light from the signal components of the photo-received interference light.

8. The apparatus according to claim 7, wherein the calculation part obtains the respective autocorrelation signal components of the object light and the reference light based on interference intensity on the photodetector having a plurality of angular frequencies by which contributions of signals from the object are made zero.

9. The apparatus according to claim 8, wherein the calculation part obtains the angular frequencies by which the contributions of the signals from the object are made zero, based on an unequal optical path difference between the object light and the reference light.

10. The apparatus according to claim 9, wherein the calculation part,
with respect to the signal components of the photo-received interference light from which the respective autocorrelation signal components of the object light and the reference light have been subtracted, obtains signal components with a phase changed by 90 degrees based on the unequal optical path difference between the object light and the reference light, and
performs Fourier transformation or inverse Fourier transformation on a combination of the signal components of the photo-received interference light from which the respective autocorrelation signal components of the object light and the reference light have been subtracted, and the obtained signal components where the phase is changed.

11. The apparatus according to claim 7, wherein
the interference/dispersion/photo-receiving optical system disperses the object light and the reference light separately to be photo-received on the photodetector, and
the calculation part takes the respective signal components of the photo-received object light and reference light as the respective autocorrelation signal components.

12. The apparatus according to claim 7, wherein
the object is an eye, and
the calculation part obtains at least one of a sectional image, a surface shape and a depth dimension of the eye as the depth information of the object.
